(19) United States
(12) Patent Application Publication
(10) Pub. No.: US 2005/0058671 A1
(43) Pub. Date: Mar. 17, 2005

(54) Dietary supplement and method for treating digestive system-related disorders

(76) Inventors: Peter M.J. Bedding, Devon (GB); Franklin L. Pelligrini, Streetsboro, OH (US); Scott C. Anderson, Hudson, OH (US); John Hall, Hudson, OH (US)

Correspondence Address: REINHART BOERNER VAN DEUREN S.C.;ATTN: LINDA GABRIEL, DOCKET COORDINATOR, 1000 NORTH WATER STREET, SUITE 2100, MILWAUKEE, WI, 53202, US

(21) Appl. No.: 10/947,598

(22) Filed: Sep. 22, 2004

Related U.S. Application Data
(63) Continuation-in-part of application No. 10/435,367, filed on May 9, 2003.
(63) Continuation-in-part of application No. 10/802,342, filed on Mar. 17, 2004.

(51) Int. Cl. A61K 31/715; A23K 1/175; A61K 35/78

(52) U.S. Cl. 424/400; 514/54; 424/750

(57) ABSTRACT

A dietary supplement and methods for the manufacture and administration of the same are disclosed for the treatment and/or prevention of digestive disorders and digestive tract-related disorders. The dietary supplement of the present invention is orally administrable, and may be compounded either in a solid form, in a paste form, in a granular form, in a powder form, or in a liquid form. The ingredients of the dietary supplement of the present invention when combined provide a synergistic efficacy which greatly exceeds the sum of the efficacies of the individual ingredients, making the dietary supplement highly effective in the treatment and/or prevention of digestive disorders and digestive tract-related disorders.

### BACKGROUND OF THE INVENTION

### Identification of Related Applications

This patent application is a continuation-in-part of copending U.S. patent application Ser. No. 10/435,367, filed on May 9, 2003, entitled “Dietary Supplement and Method for the Treatment and Prevention of Digestive Tract Ulcers in Equines and Other Animals,” which patent application is assigned to the assignee of the present invention, and which patent application is hereby incorporated herein by reference in its entirety. This patent application is also a continuation-in-part of copending U.S. patent application Ser. No. 10/802,342, filed on Mar. 17, 2004, entitled “Nutritional Product and Method for Optimizing Nutritional Uptake in Equine Foals and Other Animals,” which is in turn a continuation-in-part of the aforementioned U.S. patent application Ser. No. 10/435,367, and which patent application is also hereby incorporated herein by reference in its entirety.

### FIELD OF THE INVENTION

The present invention relates generally to dietary supplements for humans and animals, and more particularly to a novel dietary supplement for use in treating and/or preventing digestive disorders as well as ailments related to the digestive system.

It has been estimated that some form of digestive disorder affects fully one-third of the population, with maladies which are either directly or indirectly tied to the digestive system affecting a further portion of the population. These disorders may be categorized into three groups: a first group, which includes digestive disorders directly and adversely affecting the digestive tract; a second group, which includes digestive disorders which indirectly and adversely affect the digestive tract; and a third group, which includes disorders which adversely affect parts of the body other than the digestive tract but are indirectly caused or affected by the digestive system.

The first group of disorders includes peptic ulcers, ulcerative colitis, irritable bowel syndrome, diverticulitis, and Crohn's disease. Peptic ulcers are sores on the lining of the stomach of the duodenum, which is the beginning of the small intestine. Peptic ulcers are most frequently caused by bacterial infection (Helicobacter pylori), but may also be caused by long-term use of nonsteroidal anti-inflammatory agents (NSAID's) like aspirin and ibuprofen. Rarely, cancerous tumors in the stomach or in the pancreas can also cause ulcers. In the case of humans and most other animals, gastric acid is secreted in the stomach in response to eating. With an ulcer, the protective mucous coating of the stomach and duodenum is weakened, which allows the gastric acid to attack the sensitive lining of the stomach or the duodenum and cause an ulcer.

There are two solutions to the problem of peptic ulcers in humans that are utilized. The first solution is the use of antacids (typically mixtures of magnesium and aluminum hydroxide), which are administered to temporarily neutralize acid in the stomach. However, antacids are treating the symptom rather than the problem, and thus an increase in pH in the stomach that is achieved with antacids does not last indefinitely (leading to nighttime heartburn and pain).

The second known solution is the use of drugs, which are administered to inhibit the production of gastric acid. These are presently two classes of drugs which are used for this purpose: histamine type-2 antagonists such as cimetidine (available from GlaxoSmithKline under its registered trademark TAGAMET) and ranitidine (available from Pfizer under its registered trademark ZANTAC), or proton pump inhibitors such as omeprazole (available from Astra AB under its registered trademark PRILOSEC) or esomeprazole (available from Astra AB under its registered trademark NEXIUM).

These drugs are effective in curing gastric ulcers, but are very expensive. In addition, many patients for these drugs are required to take them indefinitely to prevent the ulcers from returning, which is particularly problematic in view of the fact that they often must be taken over the long term. Another profound disadvantage is that these drugs frequently cause long-term diarrhea.

The second disorder in the first group is ulcerative colitis, which is a disease which causes inflammation and ulcers in the top layers of the lining of the large intestine. While ulcerative colitis usually occurs in the rectum and the lower part of the colon, it may affect the entire colon as well as the ileum, which is the lower section of the small intestine. The inflammation causes frequent emptying of the colon, resulting in diarrhea. Ulcers form in places where the inflammation has killed the cells of the colon lining, and they bleed and produce pus and mucus.

Ulcerative colitis is usually treated with 5-ASA medications, which are a combination of sulfonamide, sulfapyridine, and salicylate and which are used to control inflammation and thereby limit the formation of ulcers. The most common of these medications is sulfasalazine (available from Pharmacia & Upjohn under its registered trademark AZULFIDINE EN). There are a number of significant side effects of 5-ASA medications, including diarrhea. Individuals who do not respond to 5-ASA medications may be treated with corticosteroids such as prednisone and hydrocortisone to reduce inflammation, which can also have significant undesirable side effects. At least 25 percent of patients having ulcerative colitis will eventually need to have their colons removed because of massive bleeding.

The third disorder in the first group is irritable bowel syndrome, which is technically not considered to be a disease but rather which is an affliction which is marked by diarrhea, constipation, and abdominal pain. Irritable bowel syndrome is caused by excessive spasms of the large intestine, and affects up to half of all patients who seek medical help for gastrointestinal problems. Frequently, patients having irritable bowel syndrome are counseled to increase their exercise level and to modify their diets (cutting gassy carbohydrates to treat bloating and belching, avoiding laxative foods to treat diarrhea, and, most commonly, increasing fiber and liquids to treat constipation). Patients not responding to this treatment may be placed on prescription medications.

The fourth disorder in the first group is diverticulitis, and its precedent condition, diverticulosis, which is the presence in the colons of small, outwardly bulging pouches called diverticula. Ten percent of people over the age of 40 and half of people over the age of 60 have diverticulosis. When these pouches become inflamed or infected, the condition is referred to a diverticulitis, which occurs in up to one-quarter of all people having diverticulosis.

While it has not been proven, it is believed that not enough fiber in the diet is a contributing factor to diverticulosis and diverticulitis, which are referred to as diverticular disease. Symptoms are abdominal pain, cramping, nausea, and constipation, and complications of diverticular disease can lead to bleeding, infections, perforations or tears, and blockages. Treatment is typically an increase in dietary fiber, antibiotics, and mild pain medications, although occasionally surgery may become necessary in severe cases.

The fifth and last disorder in the first group is Crohn's disease, which is characterized by inflammation in the small intestine, typically in the ileum or lower part of the small intestine, although the inflammation can affect any part of the digestive tract from the mouth to the anus. The inflammation results in pain and causes the intestines to empty frequently, resulting in diarrhea. The cause of Crohn's disease has not been definitively identified, but it is believed to be due to a reaction of the immune system to a virus or bacterium which causes the inflammation. Individuals with Crohn's disease tend to have abnormalities of the digestive system, but it unclear whether these abnormalities are causes of the condition or its effect.

Treatment of Crohn's disease can vary according to its severity, with 5-ASA medications such as mesalamine being commonly used to control inflammation. The mesalamine may be dispensed as capsules or tablets (available from Shire US under its registered trademark PENTASA), rectal enemas (available from Solvay under its registered trademark ROWASA), or as rectal suppositories (also available from Solvay under its registered trademark ROWASA). Side effects include nausea and diarrhea. Corticosteroids may also be used to control inflammation. Finally, immune system suppressants such as 6-mercatopurine (available from Glaxo Wellcome under its registered trademark PURINETHOL) and azathioprine (available from GlaxoSmithKline under its registered trademark IMURAN) may also be used to suppress the immune reaction which contributes to the inflammation. Side effects of immune system suppressants are well known and significant.

Incidents of Crohn's disease not responding to these treatments may be treated with infliximab (available from Centocor under its registered trademark REMICADE), which is an anti-tumor necrosis factor (TNF) substance. Antibiotics may also be used to treat any accompanying bacterial overgrowth in the small intestine. Antidiarrheal agents may also be used to relieve symptoms, and patients may need to be rehydrated with fluids and electrolytes. No dietary factors have been shown to be of use in preventing or treating Crohn's disease, although milk, alcohol, hot spices, or fiber may exacerbate the symptoms.

The second group of disorders includes mucositis, cachexia, lactose intolerance, and dietary insufficiencies in the elderly. The first two disorders in this group, mucositis and cachexia, are both encountered in patients having cancers. Mucositis is the inflammation of the mucous membranes of the digestive tract, and occurs with many patients who receive chemotherapy or radiation of areas which include any portion of the mucous membranes in the digestive tract which treatment is designed to halt rapidly growing cancers by killing cancer cells that are in the process of mitosis (cell division). The epithelial cells of mucous membranes, which also reproduce rapidly and have a short life span, are killed or severely damaged by chemotherapy or radiation, which damage may manifest itself as swelling, irritation, and ulceration of the mucosal cells lining the digestive tract, and in abdominal cramping and diarrhea.

Patients receiving chemotherapy and radiation for tumors in the head and neck develop a particular type of oral mucositis which is referred to as stomatitis, and patients being treated for small cell lung cancer develop mucositis in the esophagus which is referred to as esophagitis. Stomatitis may affect the patients' ability to eat, and in severe cases the patients' ability to talk. Mucositis and stomatitis are treated with antibiotics and antifungal medications, and patents are encouraged to drink plenty of fluids. Where mucositis-caused ulcers are present in the digestive system, various ulcer medications may also be used.

The second disorder in the second group is cachexia, which is characterized by a wasting away of the bodies of cancer patients, as well as congestive heart failure patients and HIV patients as well. Cachexia is thought to be caused by the consumption of massive amounts of glucose by cancer cells, which process the glucose by fermentation (which is inefficient) and produce lactic acid as a byproduct of this fermentation. The liver converts the lactic acid back into glucose, which consumes still more energy, and the process repeats itself. As the cancers grow, the rest of the patients' bodies waste away, causing approximately forty percent of cancer patients to die from the malnutrition which is a hallmark of cachexia.

Cachexia has been treated by low glucose diets (which may not be used in patients in end stages of cachexia malnutrition), alkalinity by the intake of cesium chloride (which depletes potassium and can cause heart attacks), and hydrazine sulfate, an Momoamine Oxidase Inhibitor (MAOI) which requires extremely careful selection of other foods ingested, and the total avoidance of foods containing tyramine (eating such foods when using an MAOI can cause significant increases in blood pressure and heart rate, as well as severe headaches).

The third disorder in the second group is lactose intolerance, which is caused by a deficiency of lactase, an enzyme which is required to absorb and digest lactose (the sugar in milk and other dairy products). Undigested lactose in the digestive system lingers in the colon and ferments, causing abdominal pain, bloating, gas, and diarrhea. Over ten percent of Americans are lactose intolerant, and as many as 75 percent of people of African descent and Native Americans, and 90 percent of people of Asian descent are lactose intolerant. Lactose intolerance is primarily treated by diet, with lactose intolerant people simply avoiding dairy products. Lactase enzymes are also available in tablet or liquid form and may be ingested with the dairy products.

The fourth and last disorder in the second group is dietary insufficiencies in the elderly, which are characterized by malnutrition and a decrease in bone density. Other characteristics of this disorder are reduced tone of intestinal muscles, a decreased immune system, and faster than normal passage of food through the digestive system, a condition often accompanied by diarrhea. Since the elderly are often less active, dietary insufficiencies also commonly result in constipation.

The third group of disorders includes diabetes and multiple sclerosis and other neurodegenerative diseases. Diabetes is characterized by the inability to properly process glucose, a sugar which the body uses for energy. As a result, the glucose stays in the blood, resulting in high levels of blood glucose, while the cells of the body are starved for glucose. Type 1 diabetes requires insulin therapy, since the pancreas of type 1 diabetics does not make sufficient insulin to process glucose. Type 2 diabetes occurs when the pancreas makes sufficient insulin, but the body has difficulty using the insulin.

Type 2 diabetes may be treated with natural therapies, and both types of diabetes show some responsiveness to vitamins, minerals, amino acids, and other supplements. Vitamins used to treat diabetes include vitamins E, C, B6, B12, Biotin, and Niacin. Minerals used include chromium, manganese, magnesium, vanadium, potassium, and zinc. Amino acids include alpha-lipoic acid (ALA) and taurine. Other supplements potentially proving useful for diabetes include coenzyme Q10 (CoQ10), inositol, and evening primrose oil.

The other disorder in the third group is multiple sclerosis and other neurodegenerative diseases. Multiple sclerosis (MS) is a progressive disease caused by the destruction of the myelin sheath that surrounds nerve cells in the brain and the spinal cord. Symptoms vary in severity from numbness to paralysis and incontinence. Traditional treatment was with medications and physical therapy. More recently, nutritional treatments including low-saturated-fat diets, antioxidant nutrients including vitamins C and E, selenium, beta-carotene, and foods high in essential fatty acids have been demonstrated to be effective.

It is accordingly the primary objective of the present invention that it efficaciously treat digestive tract disorders as well as digestive tract-related disorders in humans and, potentially, in other animals as well. In addition to having the effect of efficaciously treating digestive tract disorders and digestive tract-related disorders, it is a related objective of the dietary supplement of the present invention that it demonstrate efficacy in the prevention of such digestive tract disorders and digestive tract-related disorders as well. It is a further objective that a single dietary supplement be effective in treating and/or preventing both a wide variety of digestive tract disorders and a number of digestive tract-related disorders as well.

It is an additional objective of the dietary supplement of the present invention that it consist entirely of safe and natural ingredients rather than drugs. It is a still further objective of the dietary supplement of the present invention that it be orally administrable, thereby making its administration a simple matter. It is a related objective of the dietary supplement of the present invention that it be able to be compounded either in a paste form, in a solid form, or in a form which is suitable to be added to liquids for delivery. It is a further related objective of the dietary supplement of the present invention that it be easy and convenient both to ship and to store.

The dietary supplement of the present invention must also be both stable and have a commercially acceptable shelf life, and it should also require no special care to be provided by the user throughout its shelf life prior to usage. In order to enhance the market appeal of the dietary supplement of the present invention, it should also be relatively inexpensive when compared to previously known methods of treatment of digestive tract disorders and digestive tract-related disorders to thereby afford it the broadest possible market. Finally, it is also an objective that all of the aforesaid advantages and objectives of the dietary supplement of the present invention and its method of administration be achieved without incurring any substantial relative disadvantage.

### SUMMARY OF THE INVENTION

The disadvantages and limitations of the background art discussed above are overcome by the present invention. With this invention, a novel dietary supplement that is formulated to treat and/or prevent a number of digestive tract disorders and a number of digestive tract-related disorders as well is provided. Through the periodic administration of this dietary supplement to humans or other animals in accordance with methods taught by the present invention, a number of digestive tract disorders and a number of digestive tract-related disorders are effectively treated and/or prevented in such humans and other animals. As will rapidly become apparent to those skilled in the art, the dietary supplement of the present invention is much more than the sum of its ingredients, with the combination of ingredients yielding a synergistic result substantially more efficacious than the results which would be produced if each of the ingredients acting by itself were provided to humans or other animals.

In its basic form, the dietary supplement of the present invention includes three principal components, each of which provides a beneficial effect on health which is facilitated by the inclusion of a particular ingredient or a mixture of ingredients in the dietary supplement. The first principal ingredient is a polar lipid supplement which preferably contains significant amounts of polar lipids and antioxidants. The polar lipid supplement acts as an emulsifier which will facilitate the absorption of water-soluble and fat-soluble nutrients (and drugs) to into the bloodstream. The polar lipid supplement also is high in polar lipids which protect and strengthen the intestinal and tissue of the digestive system and augment the protective effect of mucus in the digestive tract. Polar lipids also form the membranes of neurons and their sheathing.

In the preferred embodiment, the polar lipid supplement contains at least a substantial portion of oat oil, which contains a high concentration of polar lipids and antioxidants. Optionally, different phases of oat oil may be blended in order to vary the amount of polar lipids contained in the polar lipid supplement, and other oils such as sunflower oil, olive oil, corn oil, or soy oil may be contained in the blend of ingredients contained in the polar lipid supplement. A form of the dietary supplement which is to be compounded in a liquid form may contain two-thirds oat oil (of which polar lipids make up approximately twenty-five percent of the oat oil) and one-third sunflower oil (since the sunflower oil is a thinner oil and does not contain a high amount of polar lipids, it may be thought of as an inert ingredient). Sunflower oil will not be included in the dietary supplement if it is to be compounded into a granular or solid form.

The second principal ingredient of the dietary supplement of the present invention is soluble beta-glucan fiber which is thought to be the most potent stimulator of the immune system. Beta-glucans are polysaccharides that also extract LDL cholesterol (the bad cholesterol) from the digested foodstuffs and lead to their excretion from the body, thereby reducing the fraction of LDL cholesterol in the bloodstream. They also sequester sugars and release them over a period of time, reducing sugar highs and lows to thereby stabilize blood sugar. Beta-glucans also slow the passage of foodstuffs through the digestive system, allowing greater absorption in the small intestine, thereby preventing carbohydrate loads in the colon which could otherwise lead to excess production of lactic acid.

In the preferred embodiment, the beta-glucan used is the soluble fiber in oats, an oligosaccharide that is found in the kernel of oats and is a powder when dried. It is a jelling agent that has an increased jelling effect when exposed to water, and also has a beneficial spreading effect that spreads the dietary supplement on the inner surface of the stomach. Alternative sources of the beta-glucan are barley, yeast, and other vegetable sources.

The third principal ingredient of the dietary supplement of the present invention is at least one, and potentially two or more, amino acids. Amino acids are nutricines which exert a beneficial effect on health rather than contributing directly to nutrition. Some amino acids increase the integrity of the digestive mucosa and are particularly beneficial to a stressed digestive system. Examples of amino acids which can be utilized in the dietary supplement of the present invention to provide this beneficial effect are threonine and glutamine, and in the preferred embodiment of the dietary supplement of the present invention both threonine and glutamine are included.

Threonine is a naturally produced essential amino acid and is an important component of the chemical pathway that creates mucin produced by the goblet cells distributed throughout the intestinal tract. By assisting metabolism and nutrient absorption, threonine contributes to a smoothly functioning digestive tract. A deficiency of threonine slows the regeneration of the gut wall and depresses the production of mucus. Threonine is especially useful for wound healing and for treating stress, but it is also an essential link in the production of immunoglobulins. In the dietary supplement of the present invention, pure L-threonine is used as the source of threonine.

Glutamine is a muscle fuel and also supplies nitrogen to the immune cells of the intestinal mucosa, which help to prevent pathogenic organisms from entering the circulatory system. Glutamine is considered to be a conditionally essential amino acid under normal conditions, because the body can create as much as is needed without the intake of glutamine supplements. But when the digestive system is stressed—for instance by ulcers—large amounts of glutamine are consumed, and supplements may be needed to replenish the supply. In the dietary supplement of the present invention, pure L-glutamine is used as the source of glutamine.

In the preferred embodiment of the dietary supplement of the present invention, one or more additional constituents may be included. One such preferred additional constituent consists of mannan oligosaccharides (MOS), which are complex sugars that are used to bind pathogens and, at the same time, nourish beneficial bacteria. Although the mechanism is not well understood, mannan oligosaccharides seem to bind to attachment sites on pathogenic bacteria, preventing the pathogenic bacteria from binding to receptors in the enterocyte membrane. In the dietary supplement of the present invention, the mannan oligosaccharides are naturally derived from the cell wall of saccharomyces cerevisiae (brewer's yeast), a yeast extract, although other sources of mannan oligosaccharides are also acceptable. In addition to binding pathogenic bacteria, a mycotoxin absorbant also based upon saccharomyces cerevisiae may also be used to absorb or soak up mycotoxins in the colon.

Another additional constituent consists of nucleotides, which are vital components to metabolic functions which control the regulatory pathways in growth and provide immunity to diseases. Nucleotides are essential for cell division, when the chromosomes must be replicated. Nucleotides are typically created de novo, but they may be scavenged from the digesta in times of stress. A lack of dietary nucleotides will restrict both the growth and thickness of the intestinal wall. Nucleotides support nutrient uptake during growth periods, and are also critical during and after disease or tissue injury, helping to rebuild damaged tissue such as, for example, after chronic diarrhea. The nucleotides will also help in recovery after weight loss caused by disease or protein deprivation due to sickness. It should be noted that the mannan oligosaccharide component will typically contain approximately five percent nucleotides.

In order to keep the various constituents of the dietary supplement from separating, in the preferred embodiment an emulsifier is also used. One such emulsifier is guar gum (also known as guaran), a galactomannan which is extracted from the seed of the leguminous shrub Cyamopsis tetragonoloba. Guar gum is commonly used as an emulsifier, a thickener and a stabilizer. As such, it is not an active ingredient of the dietary supplement of the present invention.

The dietary supplement of the present invention can be manufactured in several different forms, which may either be taken directly as a dietary supplement or added to food or drink. The dietary supplement of the present invention may be manufactured as a solid, as a granulated solid, as a paste, or as a liquid. In order to manufacture it as a solid, a small amount of oat bran or oat flour (or substitutes therefor) are added to thicken it to food bar form. By adding a higher percentage of oat bran or oat flour while stirring the mixture, a granular form of the supplement may be manufactured. By adding still more flour, a powder form of the supplement may be manufactured. By adding more oil (oat oil, sunflower oil, or another oil), the mixture can be brought to a paste having the consistency of peanut butter. By adding still more oil, it can be made into a viscous liquid.

The dietary supplement of the present invention may be manufactured as a liquid or paste and stored in a gelatin capsule (as gelcaps), which makes for a consistent dosage of the dietary supplement. It is desirable that the dietary supplement of the present invention is taken on a regular basis, which in the preferred embodiment is daily or multiple times daily (for example, with meals) in order to maintain an optimal level of the ingredients in the digestive tract.

Upon disclosure of the dietary supplement of the present invention to those skilled in the art, they will immediately appreciate that the dietary supplement is much more than merely the sum of its ingredients. The combination of polar lipid supplement, soluble beta-glucan fiber, threonine, glutamine, mannan oligosaccharides, and nucleotides yield a synergistic result substantially more efficacious than a sum of the results which would be produced if each ingredient by itself was used. In addition to the utility of the polar lipid supplement of the present invention in treating and preventing various digestive tract disorders, the polar lipid supplement of the present invention also has utility in treating and preventing a number of digestive tract-related disorders as well. Depending upon the particular desired application of the dietary supplement of the present invention, additional constituents such as vitamins and minerals may also be added thereto.

It may therefore be seen that the present invention teaches a dietary supplement which efficaciously treats digestive tract disorders as well as digestive tract-related disorders in humans and, potentially, in other animals as well. In addition to having the effect of efficaciously treating digestive tract disorders and digestive tract-related disorders, the dietary supplement of the present invention demonstrates efficacy in the prevention of such digestive tract disorders and digestive tract-related disorders as well. The preferred embodiment of the dietary supplement of the present invention is effective in treating and/or preventing both a wide variety of digestive tract disorders and a number of digestive tract-related disorders as well.

The dietary supplement of the present invention consists entirely of safe and natural ingredients rather than drugs. The dietary supplement of the present invention is orally administrable, thereby making its dispensation a simple matter. The dietary supplement of the present invention may be compounded either in a paste form, in a solid form, or in a form which may be added to liquids for delivery. The dietary supplement of the present invention can also be packaged in a manner which makes it both easy to ship and to store.

The dietary supplement of the present invention is stable and has a long shelf life, and requires no special care to be provided by the user throughout its shelf life prior to usage. The dietary supplement of the present invention is also inexpensive relative to previously known digestive tract disorder treatments and digestive tract-related disorder treatments, thereby enhancing its market appeal and affording it the broadest possible market. Finally, all of the aforesaid advantages and objectives of the dietary supplement of the present invention and its method of administration are achieved without incurring any substantial relative disadvantage.

### DESCRIPTION OF THE DRAWINGS

These and other advantages of the present invention are best understood with reference to the drawings, in which:

FIG. 1 is a somewhat schematic drawing of a human being showing the anatomy of the human digestive tract.

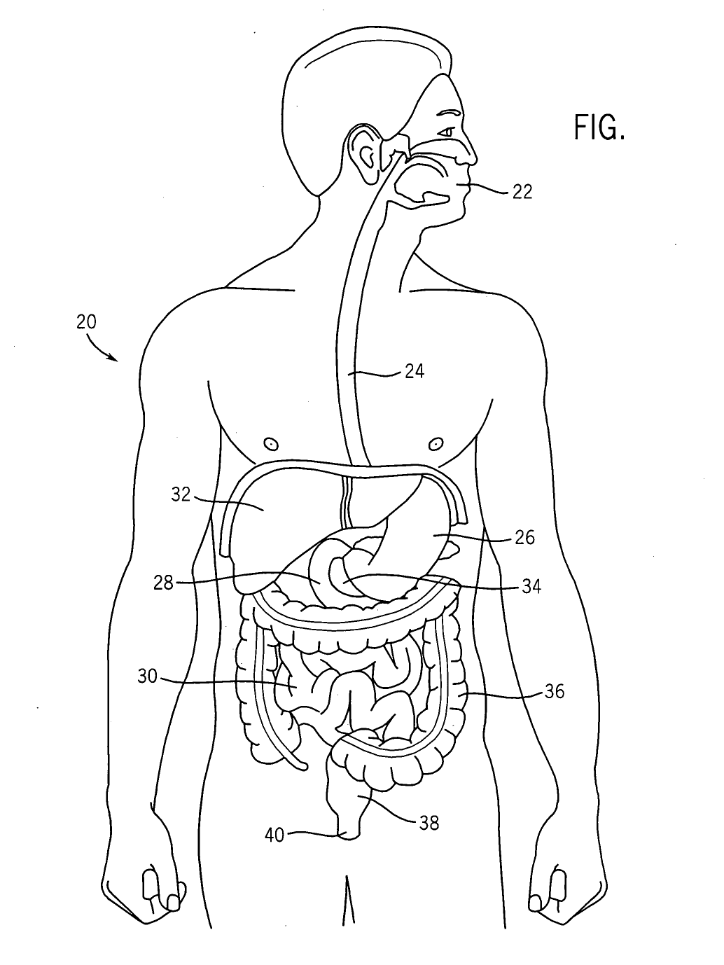

### DETAILED DESCRIPTION OF THE PREFERRED EMBODIMENT

Prior to a discussion of the nutritional product of the present invention and methods of making and administering it, it is helpful to briefly discuss the anatomy of the human digestive system. Referring to the Figure, the head and torso of a human being 20 are illustrated together with a schematic illustration of the human digestive tract. The digestive tract of a human being 20 begins at a mouth 22, and sequentially extends through an esophagus 24 and into a stomach 26. In the mouth 22, food is chewed and saliva is mixed with the food to begin digestion of carbohydrates. The food is swallowed, and passes through the esophagus 24 to the stomach 26, where pepsin assists in the digestion of protein.

From the stomach 26, the food flows through a duodenum 28, which is the first portion of a small intestine 30, where chemicals secreted by a liver 32 and a pancreas 34 enable the duodenum 28 to break down fat. From the duodenum 28, the food then moves into the small intestine 30, where the digestion process is completed. The digested food then moves to a large intestine 36, where water and sodium are removed, and then to a rectum 38. The remaining undigested solids then pass from the body through an anus 40.

In its simplest implementation, the preferred embodiment of the dietary supplement of the present invention includes three primary ingredients which are mixed together to manufacture the dietary supplement. These ingredients are a polar lipid supplement high in polar lipids and antioxidants, a soluble beta-glucan fiber which is a potent immune system stimulator, and one or more amino acids which provide a beneficial effect on the human digestive system. Each of these ingredients will be discussed separately below, together with a description of its preferred composition, alternative compositions, the preferred amount of the composition used in the dietary supplement, and the range of the amount of the composition which may be used in the dietary supplement. During the following discussion of the ingredients of the dietary supplement of the present invention, it will rapidly become apparent to those skilled in the art that the benefits achieved by the dietary supplement of the present invention are substantially greater than the sum of the individual benefits of each of the dietary supplement's ingredients.

The first primary ingredient of the dietary supplement of the present invention is a polar lipid supplement which is high in polar lipids and antioxidants. Polar lipids are emulsifiers, allowing water-soluble nutrients and fat-soluble nutrients (such as vitamins A, D, E, & K) to be easily absorbed in the bloodstream. Polar lipids include phospholipids, galactolipids, and lecithins. Polar lipids occur naturally in vegetable oils.

Polar lipids thus provide a versatile delivery vehicle for drugs and nutrients. Studies have shown that polar lipids can increase the bioavailability of dissolved drugs. In addition to their use as emulsifiers, polar lipids physically augment the protective effect of mucus in the digestive tract. They also help to reinforce the so-called “tight junctions” between the enterocytes that line the digestive tract. Finally, vegetable oils contain tocopherols and trienols, which are powerful antioxidants, sweeping free radicals out of the system.

There are a number of potential sources of polar lipids that may be used as the polar lipid supplement in the dietary supplement of the present invention. In the preferred embodiment, oat oil is used due to the fact that oat oil has more polar lipids per unit volume than any other any other polar lipid source. Oat oil is also an excellent source of antioxidants. Other oils that are also good sources of polar lipids are sunflower oil, soybean oil, olive oil, palm oil, corn oil, rapeseed oil, linseed oil, etc.

In the preferred embodiment of the dietary supplement of the present invention, the polar lipid supplement is primarily made of oat oil. Like most oils, oat oil may come from multiple extractions, with the typical extraction process crushing the oats and treating them with an extraction agent. The first phase of oat oil extracted is a thin oil. The second phase of oat oil extracted is a thick oil, which is considerably more viscous than the first phase. The third phase of oat oil extracted is a very thick oil that has the consistency of grease and which is the richest in polar lipids.

All three phases of the oat oil extraction process may be mixed, with the resulting mixture being referred to herein as oat oil. Optionally, the third phase of oat oil can be mixed with the oat oil consisting of all three phases to increase the concentration of polar lipids therein. The preferred concentration of polar lipids in the oat oil used in the dietary supplement of the present invention is approximately twenty-five percent. While there are a variety of sources for oat oil, a commercial source for the third phase of oat oil is Swedish Oat Fiber AB in Gothenburg, Sweden, which manufactures such a product.

The second primary ingredient of the dietary supplement of the present invention is a soluble beta-glucan fiber which is a powerful immune system stimulator. Beta-glucans are polysaccharides that also extract LDL cholesterol (the bad cholesterol) from the digested foodstuffs and lead to their excretion from the body, thereby reducing the fraction of LDL cholesterol in the bloodstream. They also sequester sugars and release them over a period of time, reducing sugar highs and lows to thereby stabilize blood sugar. Beta-glucans are also high in fiber content, and therefore act to slow the passage of foodstuffs through the digestive system, preventing carbohydrate loads in the colon which could otherwise lead to excess production of lactic acid.

There are a number of potential sources of soluble beta-glucan fiber that may be used in the dietary supplement in the dietary supplement of the present invention. Beta-glucan is a jelling agent that works with gastric juices or water. In the preferred embodiment, the soluble fiber used is beta-glucan that is derived from oats. Other soluble fibers that are also good sources of beta-glucan are those derived from barley or soybeans. Beta-glucan is widely available from a large number of different suppliers, and may be milled as a flour.

The third primary ingredient of the dietary supplement of the present invention is one or more nutricines such as surfactant amino acids which exert beneficial effects on a stressed digestive system. There are two amino acids that are used in the preferred embodiment of the dietary supplement of the present invention, namely L-threonine and L-glutamine, which are both naturally produced amino acids which are produced by breaking down a protein. L-threonine is a naturally produced essential amino acid which is produced by breaking down protein. L-threonine makes up collagen, elastin, and enamel protein, assists in metabolism and assimilation, and aids the digestive system by increasing the integrity of the mucous gut membrane. L-threonine has also been observed by the inventors to have a synergistic effect with beta-glucan in further slowing motility through the stomach.

L-glutamine is a naturally produced nonessential amino acid which is produced by breaking down protein. L-glutamine is the most abundant amino acid in the bloodstream, and is primarily formed and stored in skeletal muscle and the lungs (and is the primary fuel of enterocytes, essential in their growth, reproduction, and repair). L-glutamine also increases growth hormones, and when ingested has a substantial effect on maintaining and increasing mucosal integrity, including enhancing the integrity of the mucous gut membrane. L-glutamine functions to “kick start” the formation of nucleotides, which are involved in the production of cell tissue and the maturation of the intestinal mucosa, and are directly involved in the immune processes and the energy systems. A diet deficient in glutamine will most likely also likely result in a deficiency in nucleotide formation. Thus, L-threonine and L-glutamine both act to protect the inside wall of the stomach by enhancing the integrity of the mucous gut membrane. L-threonine and L-glutamine are widely available from a large number of different suppliers, and are also powders.

These principal ingredients of the dietary supplement of the present invention thus operate in ways which are enhanced by the interaction between the ingredients: by providing polar lipids to act as an emulsifier which will enhance the absorption of water-soluble and fat-soluble nutrients (and drugs) into the bloodstream, and which also provide antioxidants; by providing a potent stimulus to the immune system, reducing the fraction of LDL cholesterol in the bloodstream, and stabilizing blood sugar; and by increasing the integrity of the digestive mucosa in a manner which is particularly beneficial to stressed digestive systems. In the preferred embodiment, additional ingredients are included in the dietary supplement of the present invention to bind and eliminate pathogenic bacteria and to absorb or soak up mycotoxins, and to support the metabolic functions which control the regulatory pathways in growth and provide immunity to diseases.

An additional active ingredient included in the preferred embodiment of the dietary supplement of the present invention is a nutricine which is a pathogenic bacteria absorbant material that attracts bacteria and passes through the digestive system together with the absorbed pathogenic bacteria in the feces. This additional constituent consists of mannan oligosaccharides (MOS), which are complex sugars that are used to bind pathogens and, at the same time, nourish beneficial bacteria. Mannan oligosaccharides bind to attachment sites on pathogenic bacteria, preventing the pathogenic bacteria from binding to receptors in the enterocyte membrane. The mannan oligosaccharides are naturally derived from the cell wall of saccharomyces cerevisiae (brewer's yeast), a yeast extract, although other sources of mannan oligosaccharides are also acceptable.

In the preferred embodiment, the pathogenic bacteria absorbant material that attracts bacteria and passes through the digestive system together with the absorbed pathogenic bacteria is a pathogenic bacteria absorbant such as the material marketed under the trademark SAFMANNAN by S.I. Lesaffre, Cedex, France. Other pathogenic bacteria absorbant nutricines that could instead be used include the material marketed under the trademark BIOSAF by S.I. Lesaffre, the material marketed under the trademark BIO-MOS by Alltech, Inc., in Nicholasville, Ky., as well as any other mannan oligosaccharide (complex mannose sugars derived from the cell wall of yeast).

Optionally, a mycotoxin absorbant also based upon saccharomyces cerevisiae may also be used to absorb or soak up mycotoxins in the colon. One such mycotoxin absorbant nutricine is a material marketed under the registered trademark MYCOSORB by Alltech, Inc. Other mycotoxin absorbant nutricines that could instead be used include the material marketed under the trademark MYCOFIX PLUS by Biomin Distribution, Inc. and the material marketed under the trademark D-MYCOTOC by Kanzy Medipharm, Inc.

An optional active ingredient which may be included in the dietary supplement of the present invention consists of a supplement which contains nucleotides, which are vital components to metabolic functions which control the regulatory pathways in growth and provide immunity to diseases. Nucleotides, which are building blocks of DNA or RNA consisting of a nitrogenous base, a phosphate molecule, and a sugar molecule (deoxyribose in DNA and ribose in RNA). Depending upon the sugar, the nucleotides are called deoxyribonucleotides or ribonucleotides. Millions of nucleotides are linked to form a DNA molecule (and thousands of nucleotides are linked to form an RNA molecule).

There are several sources for nucleotides, the best of which are derived from brewer's or baker's yeast. Two sources of nucleotide-containing supplements are S.I. Lesaffre and Alltech, Inc. The Lesaffre product is marketed under the trademark YEAST CELL EXTRACT (2006), and contains approximately fifteen percent nucleotides. The Alltech product is marketed under the registered trademark NUPRO, and contains between five and seven percent nucleotides. Generally, nucleotide-containing supplements having higher nucleotide levels are preferred over products having lower nucleotide levels. Nucleotides are also contained in mannan oligosaccharide, which typically contains approximately five percent nucleotides.

The balance of the protective and the invasive factors of the intestines determines the health of the gut. Maintenance of the mucosal bloodflow is one of the most critical and important protective factors, and is believed to be enhanced by nucleotides. The gut wall have a number of minute finger-shaped processes of the mucous membrane called villi that serve in the absorption of nutriments, with crypts located between adjacent villi. Proper nutritional uptake, the height of the villi, and increased mucosal bloodflow are all related, and reduced mucosal bloodflow will result in shortened villi and shallower crypts, which in turn results in a decreased level of nutritional uptake. Animal studies have demonstrated that dietary nucleotides increase villi height and mucosal bloodflow, which in turn increases the uptake of nutrients into the body and the effectiveness of other nutritional elements.

Finally, there is a non-active ingredient which is added to the dietary supplement of the present invention as an emulsifier in order to prevent its constituents from separating. The emulsifier used in the dietary supplement in the preferred embodiment is guar gum, which also has thickening and stabilizing properties. Other emulsifiers having appropriate properties could be used instead of the guar gum, such as carrageenen and agar.

Those skilled in the art will immediately appreciate that the dietary supplement of the present invention is much more than merely the sum of its ingredients, with the combination of ingredients yielding a synergistic and highly efficacious result. For example, the polar lipid supplement acts as a spreading agent that enhances the efficacy of the soluble beta-glucan fiber and the amino acids by spreading them to cover the entire digestive tract. The soluble beta-glucan fiber slows down the passage of the polar lipid supplement and the amino acids, giving them both more time to provide their beneficial effects on the digestive tract. The amino acids also increase the integrity of the gut membrane, but are much more effective in combination with the polar lipid supplement than they would be without it.

The relative ranges of amounts of each of the ingredients, and their preferred amounts, will now be discussed, beginning with the nutricines. First, the polar lipid supplement will be discussed. It is apparent from the preceding discussion that the only non-solid ingredient in the dietary supplement of the present invention is the polar lipid supplement.

Since it is desirable to have approximately twenty-five percent polar lipids in the oat oil, the oat oil (which is the mixture of all three phases of oat oil), may be fortified by adding an additional amount of the third phase of oat oil, which is a thick paste, to increase the content of polar lipids to the desired percentage. The concentration of polar lipids in the oat oil may vary from approximately ten percent to thirty-five percent. If sunflower oil is to be added as a thinner to produce a liquid dietary supplement, the resulting concentration of polar lipids in the polar lipid supplement drops to approximately sixteen and two-thirds percent.

The range of amounts of the polar lipid supplement in the dietary supplement of the present invention is between approximately ten percent and sixty percent of the dietary supplement by weight. The preferred amount of the polar lipid supplement is between approximately forty percent and fifty-five percent of the dietary supplement by weight. The most preferred amount of the polar lipid supplement is approximately forty-eight percent of the dietary supplement by weight.

Next, the soluble beta-glucan fiber will be discussed. The range of amounts of soluble beta-glucan fiber is between approximately one percent and fifty percent of the dietary supplement by weight. The preferred amount of soluble beta-glucan fiber is between approximately fifteen and thirty percent of the dietary supplement by weight. The most preferred amount of soluble beta-glucan fiber is approximately twenty-three percent of the dietary supplement by weight.

The nutricines that increase the integrity of the mucous gut membrane, which in the preferred embodiment are L-threonine and L-glutamine, will be discussed. The range of amounts of L-threonine is between approximately one percent and twenty percent of the dietary supplement by weight. The preferred amount of L-threonine is between approximately five and fifteen percent of the dietary supplement by weight. The most preferred amount of L-threonine is approximately eleven percent of the dietary supplement by weight.

The range of amounts of L-glutamine is between approximately two percent and twenty percent of the dietary supplement by weight. It is believed that less than one percent of L-glutamine will result in little or no efficacious result. The preferred amount of L-glutamine is approximately five to fifteen percent of the dietary supplement by weight. The most preferred amount of L-glutamine is approximately nine percent of the dietary supplement by weight.

Next, the mannan oligosaccharide component will be discussed. The range of amounts of the mannan oligosaccharide component is between approximately one-half percent and ten percent of the dietary supplement by weight. The preferred amount of the mannan oligosaccharide component is approximately three to seven percent of the dietary supplement by weight. The most preferred amount of the mannan oligosaccharide component is approximately five percent of the dietary supplement by weight.

If a mycotoxin absorbant is included, its preferred amount is between approximately one percent and five percent by weight, with a preferred amount of approximately two percent.

Next, the nucleotides will be discussed. The range of amounts of nucleotide-containing supplement is between approximately zero percent and ten percent of the dietary supplement by weight. The preferred amount of nucleotide-containing supplement is approximately one to five percent of the dietary supplement by weight. The most preferred amount of nucleotide-containing supplement is approximately one percent of the dietary supplement by weight. The preferred amount of actual nucleotides (as opposed to the nucleotide-containing supplement) in the dietary supplement of the present invention is between approximately 0.01 percent and 1.5 percent by weight. Note that additional nucleotides are contained in the mannan oligosaccharide supplement, which contain approximately twenty percent nucleotides by weight.

Finally, for the preferred embodiment, the relative weight of the guar gum is approximately three percent of the dietary supplement by weight.

The dietary supplement of the present invention can be manufactured as a solid, as a granulated solid, as a powder, as a paste, or as a liquid. In order to manufacture it as a solid, a small amount of oat bran or oat flour (or substitutes therefor) are added to thicken it to food bar form. It may be added to additional ingredients to make a standard size health bar. By adding a higher percentage of oat bran or oat flour while stirring the mixture, a granular form of the supplement may be manufactured. This granular form can be sprinkled on cereal or fruit, or added to a liquid. By adding still more flour while stirring the mixture, a powder form of the supplement may be manufactured.

By adding more oil (oat oil, sunflower oil, or another oil), the mixture can be brought to a paste having the consistency of peanut butter. In the paste form, the dietary supplement of the present invention may be stored in gelatin capsules (as liquid-filled softgel capsules), which also provide for a consistent dosage of the dietary supplement. By adding still more oil, it can be made into a viscous liquid which can be taken by spoon.

It is desirable that the dietary supplement of the present invention is taken on a regular basis, which in the preferred embodiment is daily in order to maintain an optimal level of the ingredients in the digestive tract. The preferred dosage is between approximately one-half teaspoon and approximately three tablespoons daily. The dietary supplement of the present invention can be taken orally at least once, and possibly twice or three times, daily. It is preferably taken with meals by those individuals who have lactose intolerance or diabetes.

The weight of the dietary supplement varies according to its form, with the paste form having a specific density of approximately 0.8, and the granular or flour forms having a specific density of between 0.5 and 0.6. Thus, the preferred dosage of the dietary supplement of the present invention may vary between approximately one gram and approximately thirty grams per day.

Since the dietary supplement of the present invention increases the absorption of nutrients in the bloodstream and slows the motility of foodstuffs through the digestive tract, it will be appreciated by those skilled in the art that by orally administering a medication in conjunction with the administration of the dietary supplement, the medication will also spend more time in the digestive tract. This will increase the absorption of the medication, and will likely thereby act to enhance the therapeutic effect of the medication. If desired, the medication can be administered at the same time the dietary supplement is administered, or even mixed or suspended in the dietary supplement prior to administration of the dietary supplement.

Upon disclosure of the dietary supplement of the present invention to those skilled in the art, they will immediately appreciate that the dietary supplement is much more than merely the sum of its ingredients. The combination of polar lipid supplement, soluble beta-glucan fiber, threonine, glutamine, mannan oligosaccharides, and nucleotides yield a synergistic result substantially more efficacious than a sum of the results which would be produced if each ingredient by itself was used. In addition to the utility of the polar lipid supplement of the present invention in treating and preventing various digestive tract disorders, the polar lipid supplement of the present invention also has utility in treating and preventing a number of digestive tract-related disorders as well. Depending upon the particular desired application of the dietary supplement of the present invention, additional constituents such as vitamins and minerals may also be added thereto.

Examples of vitamins which could be added include vitamins B6, B12, Biotin, C, E, and Niacin. Examples of mineral micronutritional additives which may be added include chromium, copper, magnesium, manganese, potassium, selenium, vanadium, and zinc. Other amino acids such as alpha-lipoic acid (ALA) and taurine may also be added. Other supplements could be added, such as, for the example of a supplement targeted at diabetes, coenzyme Q10 (CoQ10), inositol, and evening primrose oil. Those skilled in the art will appreciate that custom formulas could be made for application with specific digestive system and digestive system-related disorders.

### Treatment of Ulcers

This application of the dietary supplement of the present invention was originally based upon research with equine patients who, like humans, are monogastric and are subject to intestinal ulcers under conditions that include sporadic eating or high stress. The dietary supplement includes soluble beta-glucan fiber, which adds bulk and slows the digestive process, buffering the gastric juices in the stomach; soluble beta-glucan fiber is a powerful immune system stimulant. It also includes polar lipids that coat the intestinal surface and improve the impermeability of the enterocytes lining the gut. Glutamine and threonine are known to be important in the creation of mucus, which provides further protection from acids, peptides, and pathogens throughout the intestines.

### Treatment of Colitis

This application of the dietary supplement of the present invention can help people with colitis overcome the debilitating aspects of this painful gastric syndrome. The soluble beta-glucan fiber moderates the absorption of nutrients, helping to reduce shocks to a sensitive digestive system. In addition, soluble beta-glucan fiber helps to add bulk to the digesta, reducing both diarrhea and constipation. The addition of polar lipids to the formula helps to coat the lining of the digestive tract, making it less permeable to the acids and pathogens implicated in this disease.

### Treatment of Irritable Bowel Syndrome

This application of the dietary supplement of the present invention can help people with irritable bowel syndrome overcome the debilitating aspects of this painful affliction. The soluble beta-glucan fiber moderates the absorption of nutrients, helping to reduce shocks to a sensitive digestive system. In addition, soluble beta-glucan fiber helps to add bulk to the digesta, reducing both diarrhea and constipation. The addition of polar lipids to the formula helps to coat the lining of the digestive tract, making it less permeable to the acids and pathogens implicated in this affliction.

### Treatment of Diverticulitis

Diverticulitis is caused by the entrapment of food particles in small intestinal pockets or diverticula. This application of the dietary supplement of the present invention is based on the observation that intestinal flow is improved by the presence of soluble beta-glucan fiber in the diet. Soluble beta-glucan fiber helps to slow transit time, helping the body to better digest food. The soluble beta-glucan fiber forms a gel, helping to sequester food particles, and creating a bolus that has less chance of leaving particles behind. In addition, the formula contains polar lipids that help to coat the digestive system, improving its impermeability and making it more slippery. This helps to keep particles from snagging and collecting in the diverticula.

### Treatment of Crohn's Disease

The dietary supplement of the present invention can help people with Crohn's disease, which is caused by leakiness of the epithelial barrier of the gut. This application of the dietary supplement of the present invention is based on the observation that polar lipids coat the digestive tract and help to increase the integrity of the intestinal epithelium. Glutamine and threonine in the formula contribute to mucus production and improve the health of stressed enterocytes. Glutamine is also known to reduce the leakiness of intestinal tissue, which if untreated can lead to infection by gram-negative bacteria. The dietary supplement of the present invention also includes soluble beta-glucan fiber that stimulate the immune system, helping to further guard against pathogens that manage to breach the cellular gut wall.

### Treatment of Mucositis and Stomatitis

This application of the dietary supplement of the present invention can reduce or eliminate stomatitis and mucositis induced by radiation or chemotherapy. This application is based on evidence that the formula can help people overcome the nausea, bleeding, swelling, pain, and diarrhea that typically accompanies many cancer therapies. These therapies aim to halt a rapidly growing cancer by killing cells that are in the process of mitosis (cell division). As a side effect, rapidly dividing epithelial cells are also killed resulting in stomatitis and progressing to a more severe syndrome called mucositis.

Epithelial cells are the body's interface with the world, and they are continually being renewed by the process of mitosis. Thus, most cancer therapies also harm epithelial tissues, especially the cells lining the intestines and other mucus membranes. With their defenses down, these patients are also at an increased risk of infection. Chemotherapy depletes glutamine, and this formula helps to redress that imbalance. Glutamine taken orally can significantly reduce the duration and severity of mucositis during and after radiation therapy. It has also been shown that glutamine can reduce the effects of mucositis during bone-marrow transplantation.

The embodiment of the dietary supplement of the present invention for this application may include a higher percentage of glutamine—up to twenty percent (five grams per dose). As well as glutamine, the formula includes threonine, which is essential to the production of mucus, as well polar lipids that can help the compromised tissues fight off the effects of bacteria and gastric acids that can make a patient sick. Polar lipids, in particular galactolipids, are known to increase the bioavailability of the glutamine in the formula by up to five times, allowing the higher dose of glutamine to act like a dose five times higher, thus lowering the total amount of the supplement required. This is an important aspect of the dietary supplement of the present invention, since the patient may find eating or drinking to be difficult. Polar lipids are typically extracted from oat flour with an alcohol process. For this formula, the alcohol will be removed under vacuum, since alcohol is contra-indicated for mucositis.

In addition, this application of the dietary supplement of the present invention incorporates small doses of zinc and vitamin B-12, which are also known to help relieve the symptoms of mucositis. Oat oil used in the formulation also includes vitamin E, or tocopherol, which is also known to help heal injured tissue. The addition of soluble beta-glucan fiber, in the form of concentrated oat flour, stimulates the immune system to stave off opportunistic infections. Thus, the dietary supplement of the present invention can help people recover faster from cancer therapies, and possibly increase the recovery rate.

The formula for this application is a paste that can eaten as is or spread on bread like peanut butter. For patients that are too sick to eat, the granular form can be mixed with water or milk to create a liquid that can be drunk.

### Treatment of Cachexia

This application of the dietary supplement of the present invention can help a person with wasting disease, or cachexia, to put on weight and thus speed their recovery. It has been established that glutamine is helpful for HIV patients who are cachexic. Glutamine is an abundant amino acid, but in times of stress, the digestive system may not get enough of it to properly maintain its high growth rate. The dietary supplement of the present invention contains glutamine along with polar lipids to improve the bioavailability of this amino acid. It also includes threonine, which is an integral part of the mucus-generating pathway. Mucus, in turn, helps to maintain the barrier between the body and the digesta. Enhancing this barrier may help to prevent the loss of blood or sera that can contribute to wasting.

### Treatment of Lactose Intolerance

This application of the dietary supplement of the present invention can reduce or eliminate the uncomfortable side effects of lactose intolerance. This application is based upon evidence that the formula can help people to tolerate milk products that typically would sicken them. It is believed that substances in the formula help to slow the transit of the digesta, helping people with low levels of lactase to have more time for digestion of lactose-containing foods.

### Treatment of Dietary Insufficiencies in the Elderly

This application of the dietary supplement of the present invention can help the elderly deal more effectively with a compromised digestive system. This application is based upon evidence that glutamine and soluble beta-glucan fiber can help to increase intestinal muscle tone and stimulate the immune system. Soluble beta-glucan fiber is known to slow the transit of digesta, which moderates the food bolus, allowing water to be resorbed and avoiding diarrhea while at the same time providing a bulking agent that minimizes constipation. The result is better tone, more predictable elimination and less gastric distress.

### Treatment of Diabetes

This application of the dietary supplement of the present invention can help diabetics to reduce or even eliminate their insulin. This application is based upon evidence that soluble beta-glucan fiber can slow the transit of digesta, which lowers the glycemic index of the meal. Soluble beta-glucan fiber is a dietary fiber that can absorb and sequester starches and sugars, releasing them over a longer time. A low glycemic index is the result, providing a slow release of sugars to the blood. This reduces the need for insulin to respond to large swings in blood sugar levels, allowing people with a challenged pancreas to deal better with their nutrition. People with only a slight diabetic tendency may be able to forgo daily injections of insulin as long as they continue to use the dietary supplement of the present invention.

### Treatment of Multiple Sclerosis

### and other Neurodegenerative Diseases

This application of the dietary supplement of the present invention can help people with multiple sclerosis or other myelin-deficient diseases, such as amyotrophic lateral sclerosis (ALS) or even potentially Alzheimer's. This application is based upon the observation that galactolipids are an essential component of the myelin sheath that insulates nerve cells, allowing low-noise communication from cell to cell. The myelin sheath is produced by cells called oligodendrocytes, which send out processes that flatten and wrap around neuronal axons. the dietary supplement of the present invention also incorporates vitamin E (tocopherol), which is indicated as potential contributor to the health of oligodendrocytes.

It may therefore be appreciated from the above detailed description of the preferred embodiment of the present invention that it teaches a dietary supplement which efficaciously treats digestive tract disorders as well as digestive tract-related disorders in humans and, potentially, in other animals as well. In addition to having the effect of efficaciously treating digestive tract disorders and digestive tract-related disorders, the dietary supplement of the present invention demonstrates efficacy in the prevention of such digestive tract disorders and digestive tract-related disorders as well. The preferred embodiment of the dietary supplement of the present invention is effective in treating and/or preventing both a wide variety of digestive tract disorders and a number of digestive tract-related disorders as well.

The dietary supplement of the present invention consists entirely of safe and natural ingredients rather than drugs. The dietary supplement of the present invention is orally administrable, thereby making its dispensation a simple matter. The dietary supplement of the present invention may be compounded either in a paste form, in a solid form, or in a form which may be added to liquids for delivery. The dietary supplement of the present invention can also be packaged in a manner which makes it both easy to ship and to store.

The dietary supplement of the present invention is stable and has a long shelf life, and requires no special care to be provided by the user throughout its shelf life prior to usage. The dietary supplement of the present invention is also inexpensive relative to previously known digestive tract disorder treatments and digestive tract-related disorder treatments, thereby enhancing its market appeal and affording it the broadest possible market. Finally, all of the aforesaid advantages and objectives of the dietary supplement of the present invention and its method of administration are achieved without incurring any substantial relative disadvantage.

Although the foregoing description of the present invention has been shown and described with reference to particular embodiments and applications thereof, it has been presented for purposes of illustration and description and is not intended to be exhaustive or to limit the invention to the particular embodiments and applications disclosed. It will be apparent to those having ordinary skill in the art that a number of changes, modifications, variations, or alterations to the invention as described herein may be made, none of which depart from the spirit or scope of the present invention. The particular embodiments and applications were chosen and described to provide the best illustration of the principles of the invention and its practical application to thereby enable one of ordinary skill in the art to utilize the invention in various embodiments and with various modifications as are suited to the particular use contemplated. All such changes, modifications, variations, and alterations should therefore be seen as being within the scope of the present invention as determined by the appended claims when interpreted in accordance with the breadth to which they are fairly, legally, and equitably entitled.

1. A dietary supplement for the treatment and prevention of digestive system and digestive system-related disorders, said digestive supplement comprising:
polar lipid supplement which contains significant amounts of polar lipids and antioxidants;
soluble beta-glucan fiber that provides at least one beneficial effect on health; and
at least one amino acid that provides at least one beneficial effect on a digestive system.

2. A dietary supplement as defined in claim 1, wherein said polar lipid supplement contains lipids of oats.

3. A dietary supplement as defined in claim 1, wherein said polar lipid supplement comprises at least one oil selected from the group consisting of oat oil, sunflower oil, soybean oil, olive oil, palm oil, corn oil, rape seed oil, and linseed oil.

4. A dietary supplement as defined in claim 1, wherein said polar lipid supplement comprises:
oat oil.

5. A dietary supplement as defined in claim 4, wherein said oat oil comprises a mixture of oat oil from all phases of the extraction process.

6. A dietary supplement as defined in claim 4, wherein said polar lipid supplement additionally comprises:
oat oil from the last phase of the extraction process, said oat oil from the last phase of the extraction process being thick and having a relatively high viscosity.

7. A dietary supplement as defined in claim 4, wherein said polar lipid supplement additionally comprises:
at least one additional oil selected from the group consisting of sunflower oil, soybean oil, olive oil, palm oil, corn oil, rape seed oil, and linseed oil.

8. A dietary supplement as defined in claim 4, wherein said oat oil comprises approximately twenty-five percent polar lipids.

9. A dietary supplement as defined in claim 1, wherein said polar lipid supplement comprises between approximately ten percent and sixty percent of said dietary supplement by weight.

10. A dietary supplement as defined in claim 9, wherein said polar lipid supplement comprises between approximately forty percent and fifty-five percent of said dietary supplement by weight.

11. A dietary supplement as defined in claim 10, wherein said polar lipid supplement comprises approximately forty-eight percent of said dietary supplement by weight.

12. A dietary supplement as defined in claim 1, wherein said soluble beta-glucan fiber is derived from oats.

13. A dietary supplement as defined in claim 1, wherein said soluble beta-glucan fiber comprises between approximately one percent and fifty percent of said dietary supplement by weight.

14. A dietary supplement as defined in claim 13, wherein said soluble beta-glucan fiber comprises between approximately fifteen and thirty percent of said dietary supplement by weight.

15. A dietary supplement as defined in claim 14, wherein said soluble beta-glucan fiber comprises approximately twenty-three percent of said dietary supplement by weight.

16. A dietary supplement as defined in claim 1, wherein said at least one amino acid comprises:
L-threonine.

17. A dietary supplement as defined in claim 16, wherein said L-threonine comprises between approximately one percent and twenty percent of said dietary supplement by weight.

18. A dietary supplement as defined in claim 17, wherein said L-threonine comprises between approximately five and fifteen percent of said dietary supplement by weight.

19. A dietary supplement as defined in claim 18, wherein said L-threonine comprises approximately eleven percent of said dietary supplement by weight.

20. A dietary supplement as defined in claim 1, wherein said at least one amino acid comprise:
L-glutamine.

21. A dietary supplement as defined in claim 20, wherein said L-glutamine comprises between approximately two percent and twenty percent of said dietary supplement by weight.

22. A dietary supplement as defined in claim 21, wherein said L-glutamine comprises between approximately five percent and fifteen percent of said dietary supplement by weight.

23. A dietary supplement as defined in claim 22, wherein said L-glutamine comprises approximately nine percent of said dietary supplement by weight.

24. A dietary supplement as defined in claim 1, additionally comprising:
a nutricine that absorbs and eliminates pathogenic bacteria in the digestive tract.

25. A dietary supplement as defined in claim 24, wherein said nutricine that absorbs and eliminates pathogenic bacteria in the digestive tract comprises:
mannan oligosaccharide (MOS).

26. A dietary supplement as defined in claim 24, wherein said nutricine that absorbs and eliminates pathogenic bacteria in the digestive tract comprises:
the material marketed under the trademark SAFMANNAN by S.I. Lesaffre.

27. A dietary supplement as defined in claim 24, wherein said nutricine that absorbs and eliminates pathogenic bacteria in the digestive tract comprises between approximately one-half percent and ten percent of said dietary supplement by weight.

28. A dietary supplement as defined in claim 27, wherein said nutricine that absorbs and eliminates pathogenic bacteria in the digestive tract comprises between approximately three percent and seven percent of said dietary supplement by weight.

29. A dietary supplement as defined in claim 28, wherein said nutricine that absorbs and eliminates pathogenic bacteria in the digestive tract comprises approximately five percent of said dietary supplement by weight.

30. A dietary supplement as defined in claim 1, additionally comprising:
a nutricine that absorbs and eliminates mycotoxins in the digestive tract.

31. A dietary supplement as defined in claim 30, wherein said nutricine that absorbs and eliminates mycotoxins in the digestive tract is based upon saccharomyces cerevisiae.

32. A dietary supplement as defined in claim 30, wherein said nutricine that absorbs and eliminates mycotoxins in the digestive tract comprises:
the material marketed under the trademark MYCOSORB by Alltech, Inc.

33. A dietary supplement as defined in claim 30, wherein said nutricine that absorbs and eliminates mycotoxins in the digestive tract comprises between approximately one percent and five percent of said dietary supplement by weight.

34. A dietary supplement as defined in claim 1, additionally comprising:
a nutricine which enhances growth and/or strengthens the immune system.

35. A dietary supplement as defined in claim 34, wherein said nutricine which enhances growth and/or strengthens the immune system comprises:
a supplement containing dietary nucleotides.

36. A dietary supplement as defined in claim 35, wherein said nucleotide-containing supplements comprises approximately one percent of said dietary supplement by weight, and provides a nucleotide concentration in said dietary supplement of between approximately 0.01 percent and approximately 1.5 percent dietary nucleotides by weight.

37. A dietary supplement as defined in claim 34, wherein said nutricine which enhances growth and/or strengthens the immune system is derived from brewer's or baker's yeast.

38. A dietary supplement as defined in claim 34, wherein said nutricine which enhances growth and/or strengthens the immune system comprises:
the material marketed under the trademark YEAST CELL EXTRACT (2006) by S.I. Lesaffre.

39. A dietary supplement as defined in claim 1, additionally comprising:
an emulsifier that prevents the constituents of said dietary supplement from separating.

40. A dietary supplement as defined in claim 39, wherein said emulsifier comprises:
guar gum.

41. A dietary supplement as defined in claim 39, wherein said emulsifier that prevents the constituents of said dietary supplement from separating comprises approximately one percent of said dietary supplement by weight.

42. A dietary supplement as defined in claim 1, additionally comprising:
a medication that is carried with the other ingredients of said dietary supplement, wherein at least one of the absorption or the therapeutic value of said medication is maximized by being taken in conjunction with said dietary supplement.

43. A dietary supplement as defined in claim 1, wherein said dietary supplement is compounded as solid food bars.

44. A dietary supplement as defined in claim 1, wherein said dietary supplement is compounded as a paste.

45. A dietary supplement as defined in claim 1, wherein said dietary supplement is compounded as a granulated solid.

46. A dietary supplement as defined in claim 1, wherein said dietary supplement is compounded as a powder.

47. A dietary supplement as defined in claim 1, wherein said dietary supplement is compounded as a liquid.

48. A dietary supplement as defined in claim 1, wherein said dietary supplement is compounded as liquid-filled softgel capsules.

49. A dietary supplement as defined in claim 1, wherein said digestive system disorders are selected from a group comprising ulcers, colitis, irritable bowel syndrome, diverticulosis, diverticulitis, Crohn's disease, mucositis, and stomatitis.

50. A dietary supplement as defined in claim 1, wherein said digestive system related disorders are selected from a group comprising cachexia, lactose intolerance, dietary insufficiencies in the elderly, diabetes, multiple sclerosis, and other neurodegenerative diseases.

51. A dietary supplement as defined in claim 1, additionally comprising:
at least one vitamin from the group consisting of vitamin B6, vitamin B12, Biotin, vitamin C, vitamin E, and Niacin.

52. A dietary supplement as defined in claim 1, additionally comprising:
at least one mineral micronutritional additive from the group consisting of chromium, copper, manganese, magnesium, manganese, potassium, selenium, vanadium, and zinc.

53. A dietary supplement as defined in claim 1, wherein taking a daily dosage of between approximately one gram and approximately thirty grams of said dietary supplement daily provides effective amounts of said polar lipid supplement, said soluble beta-glucan fiber, and said at least one amino acid.

54. A dietary supplement as defined in claim 1, additionally comprising:
a medication that is carried with the other ingredients of said dietary supplement, wherein at least one of the absorption or the therapeutic value of said medication is maximized by being taken in conjunction with said dietary supplement.

55. A dietary supplement for the treatment and prevention of digestive system and digestive system-related disorders, said digestive supplement comprising:
a polar lipid supplement which contains significant amounts of polar lipids and antioxidants;
soluble beta-glucan fiber that exerts at least one beneficial effect on health;
a first surfactant amino acid-based nutricine which assists metabolism and nutrient absorption; and
a second surfactant amino acid-based nutricine which is a muscle fuel and supports the immune function of the digestive system.

56. A dietary supplement for the treatment and prevention of digestive system and digestive system-related disorders, said digestive supplement comprising:
a polar lipid supplement which contains significant amounts of polar lipids and antioxidants;
soluble beta-glucan fiber;
L-threonine;
L-glutamine; and
a nutricine that absorbs and eliminates pathogenic bacteria in the digestive tract.

57. A dietary supplement for the treatment and prevention of digestive system and digestive system-related disorders, said digestive supplement comprising:
an effective amount of a polar lipid supplement;
an effective amount of beta-glucan;
an effective amount of L-threonine; and
an effective amount of L-glutamine.

58. A dietary supplement for the treatment and prevention of digestive system and digestive system-related disorders, said digestive supplement comprising:
a polar lipid supplement which contains a daily dosage of about 25 mg to about 4500 mg of polar lipids;
soluble beta-glucan fiber that exerts at least one beneficial effect on health; and
at least one amino acid which is capable of exerting at least one beneficial effect on a stressed digestive system.

59. A dietary supplement for the treatment and prevention of digestive system and digestive system-related disorders, said digestive supplement comprising:
a polar lipid supplement;
soluble beta-glucan fiber; and
an amino acid.

60. A method of making a dietary supplement for the treatment and prevention of digestive system and digestive system-related disorders, said method comprising:
providing a polar lipid supplement which contains significant amounts of polar lipids and antioxidants;
providing soluble beta-glucan fiber that exerts at least one beneficial effect on health;
providing at least one amino acid which is capable of exerting at least one beneficial effect on a stressed digestive system; and
mixing said polar lipid supplement, said soluble beta-glucan fiber, and said one amino acid which is capable of exerting at least one beneficial effect on a stressed digestive system together.

61. A method of making a dietary supplement for the treatment and prevention of digestive system and digestive system-related disorders, said method comprising:
providing a polar lipid supplement;
providing soluble beta-glucan fiber;
providing at least one amino acid which is capable of exerting at least one beneficial effect on a stressed digestive system;
providing L-threonine;
providing L-glutamine;
a nutricine that absorbs and eliminates pathogenic bacteria in the digestive tract; and
mixing said polar lipid supplement, said soluble beta-glucan fiber, said L-threonine, said L-glutamine, and said nutricine that absorbs and eliminates pathogenic bacteria in the digestive tract together.

62. A method for administering a dietary supplement for use in treating and preventing digestive system and digestive system-related disorders, said method comprising:
preparing an appropriately sized dose of dietary supplement comprising:
a polar lipid supplement which contains significant amounts of polar lipids and antioxidants;
soluble beta-glucan fiber that exerts at least one beneficial effect on health; and
at least one amino acid which is capable of exerting at least one beneficial effect on a stressed digestive system;
administering said dietary supplement on a regular basis; and
repeating said preparing and administering steps at least once daily.
